(19) United States
(12) Patent Application Publication
(10) Pub. No.: US 2026/0242342 A1
(43) Pub. Date: Aug. 20, 2026

(54) Crosslinking Compound, Ink Composition and Film Comprising the Same

(71) Applicant: UNIST (ULSAN NATIONAL INSTITUTE OF SCIENCE AND TECHNOLOGY), Ulsan (KR)

(72) Inventors: BongSoo KIM, Ulsan (KR); Wanho CHO, Ulsan (KR)

(73) Assignee: UNIST (ULSAN NATIONAL INSTITUTE OF SCIENCE AND TECHNOLOGY), Ulsan (KR)

(21) Appl. No.: 19/448,906

(22) Filed: Jan. 14, 2026

(30) Foreign Application Priority Data
Jan. 15, 2025 (KR) 10-2025-0006313
Jan. 14, 2026 (KR) 10-2026-0007090

(51) Int. Cl. C07D 229/02; C09D 11/037; C09D 11/50; C09K 11/06

(52) U.S. Cl. CPC C07D 229/02; C09D 11/037; C09D 11/50; C09K 11/06; C09K 2211/1044; C09K 2211/188

(57) ABSTRACT

Provided are a crosslinking compound and a film including the same.

### CROSS-REFERENCE TO RELATED APPLICATION

This application is based on and claims priority under 35 U.S.C. § 119 to Korean Patent Application Nos. 10-2025-0006313, filed on Jan. 15, 2025, and 10-2026-0007090, filed on Jan. 14, 2026 in the Ministry of Intellectual Property, Republic of Korea, the disclosures of which are incorporated by reference herein in their entirety.

### BACKGROUND

### 1. Field

The disclosure relates to a crosslinking compound and a film including the same.

This research was conducted with the support of Samsung Science & Technology Foundation (Project Number: SRFC-MA1901-51).

### 2. Description of the Related Art

Recently, films including semiconductor materials (for example, perovskite compounds) or materials applicable to semiconductor devices have been actively studied as next-generation display and lighting devices because, when introduced into light-emitting devices, the films offer advantages such as high luminous efficiency, high color purity, and suitability for solution processes.

However, since the above materials are susceptible to damage, as a solution to such issues, a technique has been proposed in which crosslinking compounds capable of crosslinking materials are introduced to form a crosslinked structure, thereby improving solvent resistance, mechanical stability, and electrical stability of films.

Therefore, there is a continuous demand for crosslinking techniques capable of maintaining the inherent optical and electrical properties of materials while precisely controlling the degree of crosslinking reactions.

### SUMMARY

Provided are a crosslinking compound having high compatibility, and a film including the same.

Additional aspects will be set forth in part in the description which follows and, in part, will be apparent from the description, or may be learned by practice of the presented embodiments of the disclosure.

According to an aspect of the disclosure, a crosslinking compound is represented by Formula 1 below:

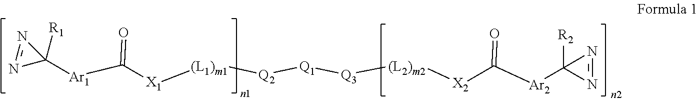

wherein, in Formula 1,
L₁ and L₂ are each independently a single bond or a C₁-C₃₀ alkylene group unsubstituted or substituted with at least one R₂₀,
m1 and m2 are each independently 0, 1, 2, 3, 4, 5, or 6,
Ar₁ and Ar₂ are each a C₅-C₆₀ carbocyclic group unsubstituted or substituted with at least one R₂₀ or a C₁-C₆₀ heterocyclic group unsubstituted or substituted with at least one R₂₀,
n1 and n2 are each independently 0, 1, 2, 3, 4, 5, 6, 7, 8, 9, or 10,
the sum of n1 and n2 is greater than or equal to 2,
Q₁ to Q₃ are each a single bond, O, S, C, C(R₃), C(R₃)(R₄), N, N(R₅), P, P(R₅)(R₆)(R₇), P=O(R₈), SO₂, a C₁-C₃₀ alkylene group unsubstituted or substituted with at least one R₂₀, or any combination thereof,
X₁ is O, S, Se, N(R₉), or C(R₉)(R₁₀),
X₂ is O, S, Se, N(R₁₁), or C(R₁₁)(R₁₂),
R₁ to R₁₂ and R₂₀ are each independently hydrogen, deuterium, —CF₃, —CCl₃, —F, —Cl, —Br, —I, a hydroxyl group, a cyano group, a C₁-C₃₀ alkyl group, a C₂-C₃₀ alkenyl group, a C₂-C₃₀ alkynyl group, a C₁-C₃₀ alkoxy group, a C₁-C₃₀ alkylthio group, a C₅-C₆₀ carbocyclic group, a C₁-C₆₀ heterocyclic group, or —Si(Q₁₁)(Q₁₂)(Q₁₃), and
Q₁₁ to Q₁₃ are each independently hydrogen, deuterium, —F, —Cl, —Br, —I, a hydroxyl group, a cyano group, a C₁-C₃₀ alkyl group, a C₂-C₃₀ alkenyl group, a C₂-C₃₀ alkynyl group, a C₁-C₃₀ alkoxy group, or a C₁-C₃₀ alkylthio group.

According to another aspect of the disclosure, a film includes the crosslinking compound.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a schematic diagram illustrating a crosslinking process in a film including a crosslinking compound according to an embodiment;

FIGS. 2A and 2B are graphs showing photoluminescence (PL) intensity, a PL peak wavelength, and a PL peak change of Example 1-1, Example 2-1, and Comparative Example 1;

FIGS. 3A and 3B are graphs showing PL intensity and a PL peak wavelength of Example 1-2, Example 2-2, and Comparative Example 2;

FIG. 4 is a graph showing a PL peak wavelength of blended films and crosslinked films of Comparative Example 1, Example 1-1, and Example 2-1;

FIGS. 5A and 5B are graphs showing PL intensity according to crosslinking time of crosslinked films in Example 3 and Comparative Example 3;

FIGS. 6A to 6C are graphs showing PL intensity properties of ink compositions of Example 4-1, Example 4-2, and Comparative Example 4;

FIGS. 7A to 7D are diagrams showing properties of crosslinked films of Example 4-1, Example 4-2, and Comparative Example 4; and

FIGS. 8A and 8B are graphs showing crosslinking efficiency according to ultraviolet (UV) absorption properties of Example 3 and Comparative Example 3 according to crosslinking time.

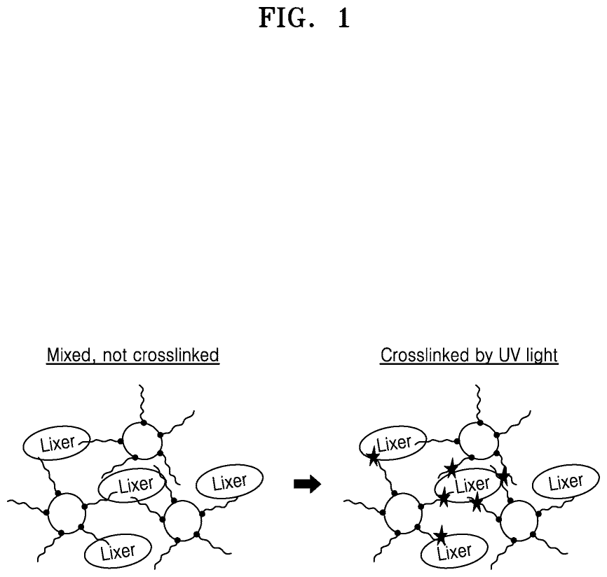

### DETAILED DESCRIPTION

Reference will now be made in detail to embodiments, examples of which are illustrated in the accompanying drawings, wherein like reference numerals refer to like elements throughout. In this regard, the embodiments may have different forms and should not be construed as being limited to the descriptions set forth herein. Accordingly, the embodiments are merely described below, by referring to the figures, to explain aspects of the description.

Hereinafter, the disclosure will be described in more detail.

In the present specification, it will be understood that the terms such as “including,” “comprising,” and “having” specify the presence of stated features or components, but do not preclude the presence or addition of one or more other features or components.

In the present specification, it will be understood that when a component such as a layer or a film is referred to as being “on” another component, the component may be directly on the other component or intervening components may be present thereon.

### [Crosslinking Compound]

According to an embodiment, a crosslinking compound is represented by Formula 1 below:

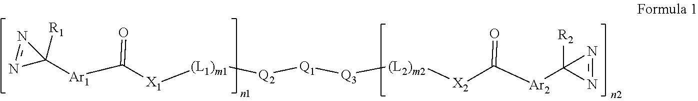

wherein, in Formula 1,
L₁ and L₂ are each independently a single bond or a C₁-C₃₀ alkylene group unsubstituted or substituted with at least one R₂₀,
m1 and m2 are each independently 0, 1, 2, 3, 4, 5, or 6,
Ar₁ and Ar₂ are each a C₅-C₆₀ carbocyclic group unsubstituted or substituted with at least one R₂₀ or a C₁-C₆₀ heterocyclic group unsubstituted or substituted with at least one R₂₀,
n1 and n2 are each independently 0, 1, 2, 3, 4, 5, 6, 7, 8, 9, or 10,
the sum of n1 and n2 is greater than or equal to 2,
Q₁ to Q₃ are each a single bond, O, S, C, C(R₃), C(R₃)(R₄), N, N(R₅), P, P(R₅)(R₆)(R₇), P=O(R₈), SO₂, a C₁-C₃₀ alkylene group unsubstituted or substituted with at least one R₂₀, or any combination thereof,
X₁ is O, S, Se, N(R₉), or C(R₉)(R₁₀),
X₂ is O, S, Se, N(R₁₁), or C(R₁₁)(R₁₂),
R₁ to R₁₂ and R₂₀ are each independently hydrogen, deuterium, —CF₃, —CCl₃, —F, —Cl, —Br, —I, a hydroxyl group, a cyano group, a C₁-C₃₀ alkyl group, a C₂-C₃₀ alkenyl group, a C₂-C₃₀ alkynyl group, a C₁-C₃₀ alkoxy group, a C₁-C₃₀ alkylthio group, a C₅-C₆₀ carbocyclic group, a C₁-C₆₀ heterocyclic group, or —Si(Q₁₁)(Q₁₂)(Q₁₃), and
Q₁₁ to Q₁₃ are each independently hydrogen, deuterium, —F, —Cl, —Br, —I, a hydroxyl group, a cyano group, a C₁-C₃₀ alkyl group, a C₂-C₃₀ alkenyl group, a C₂-C₃₀ alkynyl group, a C₁-C₃₀ alkoxy group, or a C₁-C₃₀ alkylthio group.

According to an embodiment, L₁ and L₂ may each independently be a methylene group, an ethylene group, a propylene group, or a butylene group, each unsubstituted or substituted with at least one R₂₀.

According to another embodiment, L₁ and L₂ may each independently be a methylene group unsubstituted or substituted with at least one R₂₀.

According to an embodiment, m1 may be 1, and m2 may be 1.

According to another embodiment, m1 may be 1.

According to another embodiment, m2 may be 1.

According to an embodiment, Ar₁ and Ar₂ may each independently be a benzene group, a naphthalene group, an anthracene group, a phenanthrene group, a triphenylene group, a pyrene group, a chrysene group, a cyclopentadiene group, a furan group, a thiophene group, a silole group, an indene group, a fluorene group, an indole group, a carbazole group, a benzofuran group, a dibenzofuran group, a benzothiophene group, a dibenzothiophene group, a benzosilole group, a dibenzosilole group, an aza-fluorene group, an aza-carbazole group, an aza-dibenzofuran group, an aza-dibenzothiophene group, an aza-dibenzosilole group, a pyridine group, a pyrimidine group, a pyrazine group, a pyridazine group, a triazine group, a quinoline group, an isoquinoline group, a quinoxaline group, a quinazoline group, a phenanthroline group, a pyrrole group, a pyrazole group, an imidazole group, a triazole group, an oxazole group, an isoxazole group, a thiazole group, an isothiazole group, an oxadiazole group, a thiadiazole group, a benzopyrazole group, a benzoimidazole group, a benzoxazole group, a benzothiazole group, a benzoxadiazole group, a benzothiadiazole group, a dibenzooxasiline group, a dibenzothiasiline group, a dibenzodihydroazasiline group, a dibenzodihydrodisiline group, a dibenzodihydrosiline group, a dibenzodioxin group, a dibenzooxathine group, a dibenzooxazine group, a dibenzopyran group, a dibenzodithine group, a dibenzothiazine group, a dibenzothiopyran group, a dibenzocyclohexadiene group, a dibenzodihydropyridine group, or a dibenzodihydropyrazine group, each unsubstituted or substituted with at least one R₂₀.

According to another embodiment, Ar₁ and Ar₂ may each independently be a benzene group, a naphthalene group, a phenanthrene group, a pyrene group, or an anthracene group, each unsubstituted or substituted with at least one R₂₀.

According to another embodiment, Ar₁ and Ar₂ may each independently be a benzene group unsubstituted or substituted with at least one R₂₀.

According to an embodiment, X₁ may be O, N(R₉), S, or Se.

According to another embodiment, X₁ may be O or S.

According to another embodiment, X₁ may be O.

According to an embodiment, X₂ may be O, N(R₁₁), S, or Se.

According to another embodiment, X₂ may be O or S.

According to another embodiment, X₂ may be O.

According to an embodiment, X₁ and X₂ may each independently be O or S.

According to an embodiment, Q₁ may be C, and Q₂ and Q₃ may each independently be a single bond.

According to an embodiment, Q₁ may be O, and Q₂ and Q₃ may each independently be a combination of C(R₃)(R₄) and C.

According to another embodiment, Q₁ may be O, and Q₂ and Q₃ may each independently be C(R₃)(R₄)—C. For example, when Q₂ and Q₃ are each independently C(R₃)(R₄)—C, C(R₃)(R₄) may be bonded to Q₁ and C may be bonded to L₁; C(R₃)(R₄) may be bonded to Q₁ and C may be bonded to L₂; or both may be satisfied simultaneously.

According to an embodiment, R₁ to R₁₂ and R₂₀ may each independently be: hydrogen, deuterium, —CF₃, —CCl₃, —F, —Cl, —Br, —I, a hydroxyl group, a cyano group, a C₁-C₂₀ alkyl group, or a C₁-C₂₀ alkoxy group; a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a biphenyl group, a terphenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thienyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, benzoimidazolyl group, a benzofuranyl group, a benzothienyl group, a benzoisothiazolyl group, a benzoxazolyl group, a benzoisoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, an imidazopyrimidinyl group, an azacarbazolyl group, an azadibenzofuranyl group, an azadibenzothiophenyl group, an azafluorenyl group, or an azadibenzosilolyl group; or —Si(Q₁₁)(Q₁₂)(Q₁₃).

Q₁₁ to Q₁₃ may each independently be hydrogen, deuterium, —F, —Cl, —Br, —I, a hydroxyl group, a cyano group, a C₁-C₃₀ alkyl group, a C₂-C₃₀ alkenyl group, a C₂-C₃₀ alkynyl group, a C₁-C₃₀ alkoxy group, or a C₁-C₃₀ alkylthio group.

According to another embodiment, R₁ to R₁₂ and R₂₀ may each independently be: hydrogen, deuterium, —CF₃, —CCl₃, or a C₁-C₂₀ alkyl group; a cyclopentenyl group, a cyclohexenyl group, a cycloheptenyl group, a phenyl group, a biphenyl group, a terphenyl group, a naphthyl group, a fluorenyl group, a phenanthrenyl group, anthracenyl group, a fluoranthenyl group, a triphenylenyl group, a pyrenyl group, a chrysenyl group, or a pyridinyl group; or —Si(Q₁₁)(Q₁₂)(Q₁₃).

Q₁₁ to Q₁₃ may each independently be hydrogen, deuterium, —F, —Cl, —Br, —I, a hydroxyl group, a cyano group, a C₁-C₃₀ alkyl group, a C₂-C₃₀ alkenyl group, a C₂-C₃₀ alkynyl group, a C₁-C₃₀ alkoxy group, or a C₁-C₃₀ alkylthio group.

According to an embodiment, R₁ and R₂ may each independently be hydrogen, deuterium, —CF₃, —CCl₃, —F, —Cl, —Br, —I, a hydroxyl group, a cyano group, or a C₁-C₃₀ alkyl group.

According to another embodiment, R₁ and R₂ may each independently be —CF₃.

According to an embodiment, the crosslinking compound may be represented by Formula 2 below:

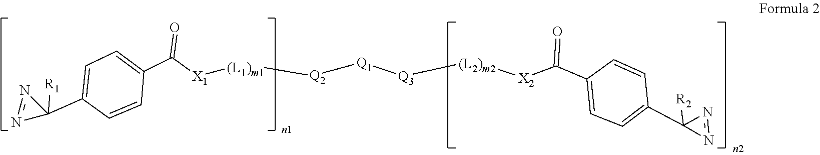

wherein, in Formula 2, L₁, L₂, m1, m2, n1, n2, Q₁ to Q₃, X₁, X₂, R₁, and R₂ may be as defined herein.

According to an embodiment, the crosslinking compound may be represented by Formula 3-1 or 3-2 below:

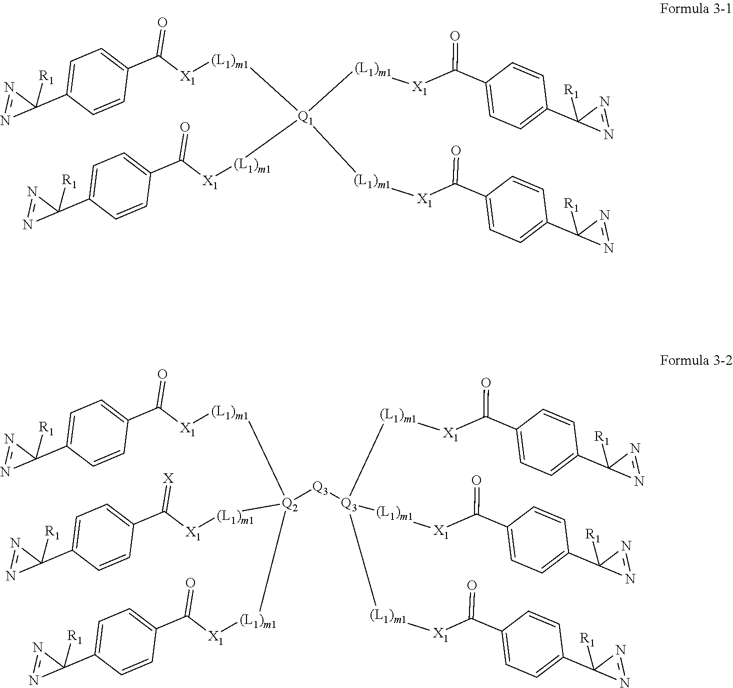

wherein, in Formulas 3-1 and 3-2, L₁, L₂, m1, m2, Q₁ to Q₃, X₁, X₂, R₁, and R₂ may be as defined herein.

According to an embodiment, in Formula 3-1, Q₁ may be C or a C₁-C₃₀ alkylene group unsubstituted or substituted with at least one R₂₀.

According to an embodiment, in Formula 3-2, Q₂ and Q₃ may each independently be C, a C₁-C₃₀ alkylene group unsubstituted or substituted with at least one R₂₀, or a combination of C(R₃)(R₄) and C.

According to an embodiment, the crosslinking compound may be represented by Formula 4-1 or 4-2 below:

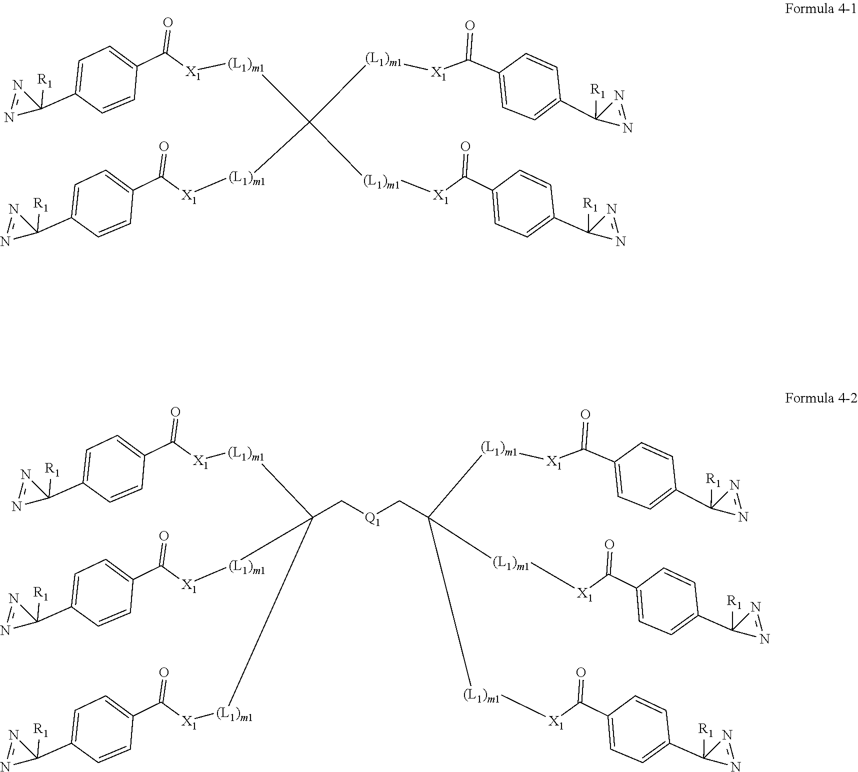

wherein, in Formula 4-1 or 4-2, L₁, m1, X₁, and Q₁ may be as defined herein.

According to an embodiment, Q₁ may be O, S, C(R₃)(R₄), or a C₁-C₃₀ alkylene group unsubstituted or substituted with at least one R₂₀.

According to an embodiment, the crosslinking compound may be crosslinking compound 1 or 2 below:

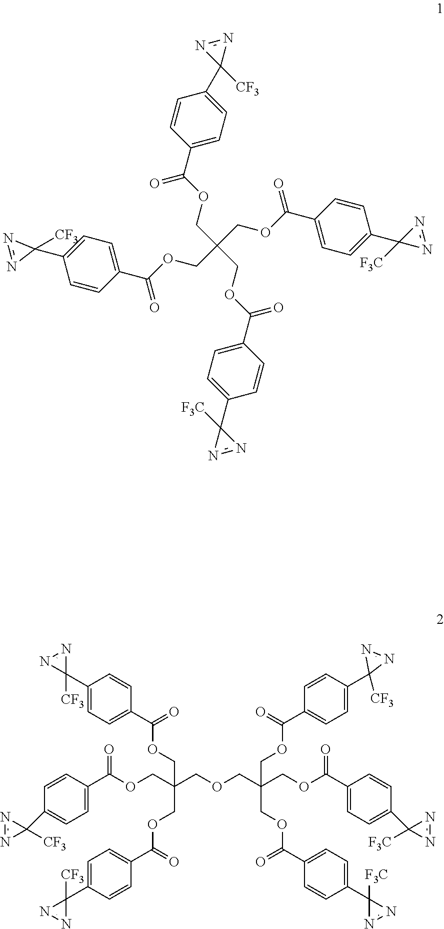 [SMILES: O=C(OCC(COC(=O)c1ccc(C2(C(F)(F)F)N=N2)cc1)(COC(=O)c1ccc(C2(C(F)(F)F)N=N2)cc1)COC(=O)c1ccc(C2(C(F)(F)F)N=N2)cc1)c1ccc(C2(C(F)(F)F)N=N2)cc1 ; O=C(OCC(COCC(COC(=O)c1ccc(C2(C(F)(F)F)N=N2)cc1)(COC(=O)c1ccc(C2(C(F)(F)F)N=N2)cc1)COC(=O)c1ccc(C2(C(F)(F)F)N=N2)cc1)(COC(=O)c1ccc(C2(C(F)(F)F)N=N2)cc1)COC(=O)c1ccc(C2(C(F)(F)F)N=N2)cc1)c1ccc(C2(C(F)(F)F)N=N2)cc1]

The crosslinking compound according to the disclosure may have excellent miscibility and compatibility with semiconductor materials, and thus even when the crosslinking compound is mixed with colloidal particles, nanocrystals, or semiconductor materials, the photoluminescence (PL) intensity of the colloidal particles, the nanocrystals, or the semiconductor materials may be maintained.

In addition, even when the crosslinking compound according to the disclosure is mixed and crosslinked with colloidal particles, nanocrystals, or semiconductor materials, the electrical and optical properties of the colloidal particles, the nanocrystals, or the semiconductor materials may be maintained during a crosslinking process, and thus the crosslinking compound according to the disclosure may have non-destructive crosslinking properties.

### [Film]

According to another aspect of the disclosure, provided is a film including the crosslinking compound.

For a description of the crosslinking compound included in the following film, reference may be made to the description of the crosslinking compound described above.

Since the film has excellent miscibility and compatibility of the crosslinking compound and undergoes a non-destructive crosslinking process, even when the crosslinking compound is mixed with colloidal particles, nanocrystals, or semiconductor materials through the crosslinking process, the properties of the colloidal particles, the nanocrystals, or the semiconductor materials may be maintained.

According to an embodiment, the film may further include a substrate.

According to an embodiment, the crosslinking compound may be applied onto the substrate.

According to an embodiment, the substrate or a base material may be selected in consideration of mechanical strength, thermal stability, surface smoothness, ease of handling, water resistance, and the like. For example, a silicon wafer or a glass substrate may be used, or a plastic film of polyethersulfone, polyacrylate, polyetherimide, polyimide, polyethylene naphthalate, or polyethylene terephthalate, or an organic substrate coated with any of these plastic films may be used.

According to an embodiment, the substrate or base material may have a single-layer structure or a multilayer structure. For example, the substrate or base material may be a single layer including a resin. For another example, the substrate or base material may have a multilayer structure including two or more layers which respectively include different types of resins. For another example, the substrate or base material may have a multilayer structure including a resin-containing layer and a functional layer, and the functional layer may be, for example, an adhesive layer, an anti-corrosion layer, an anti-reflective layer, a hard coating layer, or a combination thereof.

### [Method of Preparing Film]

According to an embodiment, a method of preparing the film may include preparing a substrate, applying an ink composition onto the substrate, spin-coating the applied ink composition, and irradiating ultraviolet (UV) light onto the spin-coated ink composition.

According to an embodiment, for a description of the substrate, reference may be made to all of the above descriptions.

According to an embodiment, for a description of the ink composition, reference may be made to all of the above descriptions.

According to an embodiment, the spin-coating of the applied ink composition may be performed at a speed of about 100 rpm to about 1,000 rpm.

According to an embodiment, the spin-coating may be performed for about 10 seconds to about 300 seconds.

According to an embodiment, the irradiating of UV light onto the spin-coated ink composition may include performing exposure at about 10 mJ/cm² to about 1,000 mJ/cm².

For a more detailed description of the method of preparing the film, reference may be made to examples.

### [Definition of Substituent]

In the present specification, a C₅-C₆₀ carbocyclic group refers to a monocyclic or polycyclic group having 5 to 60 carbon atoms and including only carbon as a ring-forming atom. The C₅-C₆₀ carbocyclic group may be an aromatic carbocyclic group or a non-aromatic carbocyclic group. The C₅-C₆₀ carbocyclic group may be a ring such as benzene, a monovalent group such as a phenyl group, or a divalent group such as a phenylene group. Alternatively, according to the number of substituents connected to the C₅-C₆₀ carbocyclic group, the C₅-C₆₀ carbocyclic group may be variously modified into a trivalent group, a tetravalent group, or the like.

In the present specification, a C₁-C₆₀ heterocyclic group refers to a group having the same structure as the C₅-C₆₀ carbocyclic group, except that the group includes, as a ring-forming atom, at least one heteroatom selected from N, O, Si, P, and S in addition to carbon (the number of carbon atoms may be 1 to 60).

In the present specification, the C₁-C₃₀ alkyl group refers to a linear or branched aliphatic hydrocarbon group having 1 to 30 carbon atoms, and specific examples thereof include a methyl group, an ethyl group, a propyl group, an isobutyl group, a sec-butyl group, a ter-butyl group, a pentyl group, an iso-amyl group, a hexyl group, a heptyl group, an n-octyl group, a 2-ethylhexyl group, and the like.

In the present specification, the C₁-C₃₀ alkylene group refers to a divalent group having the same structure as the C₁-C₃₀ alkyl group.

In the present specification, a C₂-C₃₀ alkenyl group refers to a hydrocarbon group including one or more carbon-carbon double bonds in the middle or terminus of the C₂-C₃₀ alkyl group, and specific examples thereof include an ethenyl group, a propenyl group, a butenyl group, and the like.

In the present specification, the C₂-C₃₀ alkynyl group refers to a hydrocarbon group including one or more carbon-carbon triple bonds in the middle or terminus of the C₂-C₃₀ alkyl group, and specific examples thereof include an ethynyl group, a propynyl group, and the like.

In the present specification, a C₁-C₃₀ alkoxy group refers to a monovalent group having a formula of—OA₁₀₁ (A₁₀₁ is the C₁-C₃₀ alkyl group), and specific examples thereof include a methoxy group, an ethoxy group, an isopropyloxy group, and the like.

In the present specification, a C₁-C₃₀ alkylthio group refers to a monovalent group having a formula of—SA₁₀₁ (A₁₀₁ is the C₁-C₃₀ alkyl group), and specific examples thereof include a methylthio group, an ethylthio group, an isopropylthio group, and the like.

As used herein, unless otherwise defined, the symbols “* and *′” each denote a bonding site with an adjacent atom in a corresponding formula.

Hereinafter, the disclosure will be described in more detail by way of examples. These examples are for describing the disclosure in more detail, and the scope of the disclosure is not limited by the examples.

### Examples

### Synthesis Example 1-1: Preparation of Crosslinking Compound 1 (diazirine-4Bx)

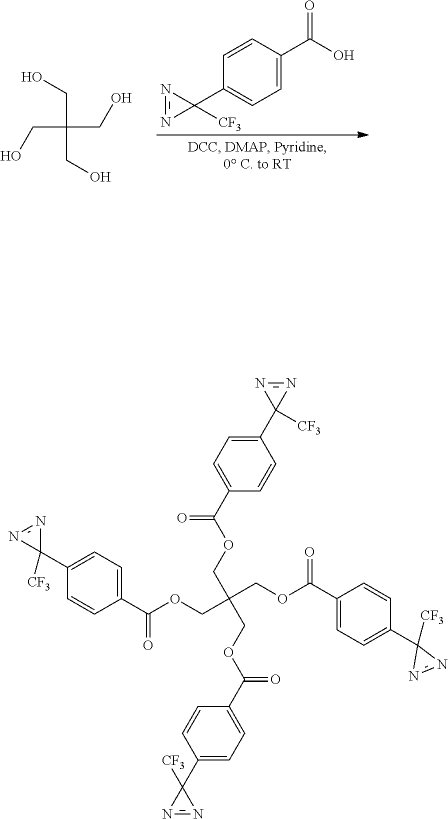 [SMILES: OCC(CO)(CO)CO ; O=C(O)c1ccc(C2(C(F)(F)F)N=N2)cc1 ; O=C(OCC(COC(=O)c1ccc(C2(C(F)(F)F)N=N2)cc1)(COC(=O)c1ccc(C2(C(F)(F)F)N=N2)cc1)COC(=O)c1ccc(C2(C(F)(F)F)N=N2)cc1)c1ccc(C2(C(F)(F)F)N=N2)cc1]

A dried magnetic bar was prepared and put into a 10 m L round-bottom flask. The round-bottom flask was vacuum-dried while being heated with a torch, and then filled with argon. In the flask, a 4-(3-(trifluoromethyl)-3H-diazirin-3-yl)benzoic acid (1,014 mg, 4.4059 mmol), pentaerythritol (100.0 mg, 0.7345 mmol), and 4-dimethylaminopyridine (DMAP) (269.0 mg, 2.2018 mmol) were dissolved in anhydrous pyridine (7.3 mL), a temperature was reduced to 0° C., and then stirring was performed for 15 minutes. Thereafter, N,N′-dicyclohexylcarbodiimide (DCC) (909 mg, 4.4055) was added, after 10 minutes, the temperature was raised to room temperature, and stirring was performed for 8 hours. The reaction was terminated, precipitated materials were filtered by using filter paper, and a filtered solution was collected. An extraction process was performed by using a separatory funnel, distilled water, and dichloromethane (30 mL). An organic layer obtained by repeating the extraction process three times was dehydrated by using anhydrous MgSO₄, and then an organic solvent was removed by using a rotary evaporator. As a result, an obtained organic material was separated by silica gel column chromatography (ethylacetate:n-hexane=1:6) to obtain a white solid product. The solid product was analyzed and had a weight of 484.6 mg and a yield of 67%. ¹H NMR (400 MHz, CDCl₃) δ=7.96-7.98 (d, 8H), 7.18-7.20 (d, 8H), 4.65 (s, 8H) ppm, ¹⁹F NMR (376 MHz, CDCl₃) δ=−65.39-64.66 (m, 12 F), ESI-MS m/z calculated: 984.1525; observed: 1007.1415 (M+Na)

### Synthesis Example 1-2: Preparation of Crosslinking Compound 2 (diazirine-6Bx)

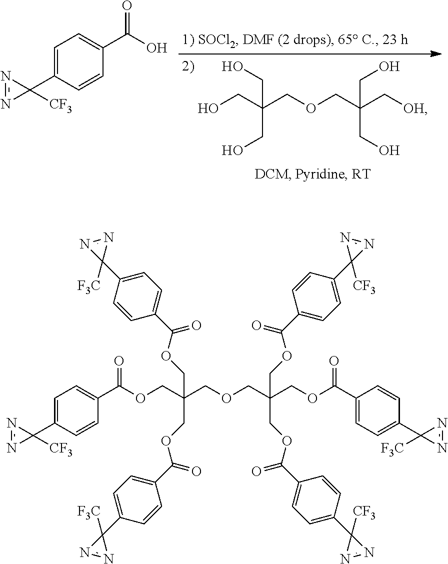 [SMILES: O=C(O)c1ccc(C2(C(F)(F)F)N=N2)cc1 ; OCC(CO)(CO)COCC(CO)(CO)CO ; O=C(OCC(COCC(COC(=O)c1ccc(C2(C(F)(F)F)N=N2)cc1)(COC(=O)c1ccc(C2(C(F)(F)F)N=N2)cc1)COC(=O)c1ccc(C2(C(F)(F)F)N=N2)cc1)(COC(=O)c1ccc(C2(C(F)(F)F)N=N2)cc1)COC(=O)c1ccc(C2(C(F)(F)F)N=N2)cc1)c1ccc(C2(C(F)(F)F)N=N2)cc1]

A dried magnetic bar was prepared and put into a 10 m L round-bottom flask. The round-bottom flask was vacuum-dried while being heated with a torch, and then filled with argon. A 4-(3-(trifluoromethyl)-3H-diazirin-3-yl)benzoic acid (2,000 mg, 8.6901 mmol) was added to the flask and vacuum-dried for 1 hour. A mixture is prepared by sequentially adding thionyl chloride (SOCl₂) (1.0 mL, 13.7863 mmol) and anhydrous dimethylformamide (DMF) (2 drops) to the flask. The mixture was heated to 65° C. and stirred for 23 hours. The mixture was cooled to room temperature, an organic solvent was removed by using a rotary evaporator, and then the mixture was additionally dried by using a vacuum pump to obtain a product. The product was diluted with anhydrous dichloromethane (DCM) (15 mL) to prepare a first mixture, which was prepared under an argon atmosphere.

A magnetic bar was prepared and put into a separate 25 mL round-bottom flask. The round-bottom flask was vacuum-dried while being heated with a torch, and then filled with argon. Dipentaerythritol (245.5 mg, 0.9656 mmol) and anhydrous pyridine (20.0 mL) were added to the flask and stirred to prepare a mixture. The previously prepared first mixture was added dropwise to the mixture to prepare a second mixture. The second mixture was stirred at room temperature for 8 hours, and then an organic solvent was removed at a temperature 40° C. by using a rotary evaporator. Accordingly, an obtained solid product was separated by silica gel column chromatography (ethylacetate:dichloromethane:n-hexane=1:2:7) to obtain a white solid product. The solid product was analyzed and had a weight of 862.0 mg and a yield of 58%. ¹H NMR (400 MHz, CDCl₃) δ=7.90-7.88 (d, 12H), 7.12-7.14 (d, 12H), 4.54 (s, 12H), 3.63 (s, 4H) ppm, ¹⁹F NMR (376 MHz, CDCl₃) δ=−64.89 (m, 18 F), ESI-MS m/z calculated: 1527.0663, observed: 1549.2438 (M+Na)

### Example 1-1: Perovskite Compound+Crosslinking Compound 1 (Diazirine-4Bx)+Decylamine Ligand

### Preparation of Ink Composition

1.5 mL of ethyl acetate was added to 1 ml of a FAPbBr₃ solution (including dodecyl amine and an oleic acid ligand), and centrifuged at a speed of 12,000 rpm for 5 minutes to precipitate particles. The precipitated particles were redispersed in toluene, in which 10 wt % of crosslinking compound 1 (10 wt % based on particles, in this case, 0.6 mg/ml) and the ligand (2 mM) were dissolved, and centrifuged at a speed of 3,750 rpm for 5 minutes to remove aggregates. A corresponding supernatant was filtered by using a 0.2 polytetrafluoroethylene (PTFE) filter.

### Preparation of Blended Film

A prepared ink composition was applied onto glass and then spin-coated at a speed of 500 rpm for 30 seconds.

### Preparation of Crosslinked Film

The prepared ink composition was applied onto glass and then spin-coated at a speed of 500 rpm for 30 seconds. Thereafter, a film was exposed to UV light through a UV irradiator (0.35 mW/cm²) with a total dose of 210 mJ/cm² to prepare a crosslinked film.

### Example 1-2: Perovskite Compound+Crosslinking Compound 1 (Diazirine-4Bx)+Tributylphosphine Oxide Ligand

### Preparation of Ink Composition

An ink composition was prepared in the same manner as in the preparation of the ink composition of Example 1-1, except that tributylphosphine oxide was used as a ligand.

### Preparation of Blended Film

The prepared ink composition was applied onto glass and then spin-coated at a speed of 500 rpm for 30 seconds.

### Preparation of Crosslinked Film

The prepared ink composition was applied onto glass and then spin-coated at a speed of 500 rpm for 30 seconds. Thereafter, a film was exposed to UV light through a UV irradiator (0.35 mW/cm²) with a total dose of 210 mJ/cm² to prepare a crosslinked film.

### Example 2-1: Perovskite Compound+Crosslinking Compound 2 (Diazirine-6Bx)+Decylamine Ligand

### Preparation of Ink Composition

1.5 mL of ethyl acetate was added to 1 ml of a FAPbBr₃ solution (including dodecyl amine and an oleic acid ligand), and centrifuged at a speed of 12,000 rpm for 5 minutes to precipitate particles. The precipitated particles were redispersed in toluene, in which 10 wt % of crosslinking compound 2 (10 wt % based on particles, in this case, 0.6 mg/ml) and the ligand (2 mM) were dissolved, and centrifuged at a speed of 3,750 rpm for 5 minutes to remove aggregates. A corresponding supernatant was filtered by using a 0.2 PTFE filter.

### Preparation of Blended Film

A prepared ink composition was applied onto glass and then spin-coated at a speed of 500 rpm for 30 seconds.

### Preparation of Crosslinked Film

The prepared ink composition was applied onto glass and then spin-coated at a speed of 500 rpm for 30 seconds. Thereafter, a film was exposed to UV light through a UV irradiator (0.35 mW/cm²) with a total dose of 210 mJ/cm² to prepare a crosslinked film.

### Example 2-2: Perovskite Compound+Crosslinking Compound 2 (Diazirine-6Bx)+Tributylphosphine Oxide Ligand

### Preparation of Ink Composition

An ink composition was prepared in the same manner as in the preparation of the ink composition of Example 2-1, except that tributylphosphine oxide was used as a ligand.

### Preparation of Blended Film

The prepared ink composition was applied onto glass and then spin-coated at a speed of 500 rpm for 30 seconds.

### Preparation of Crosslinked Film

The prepared ink composition was applied onto glass and then spin-coated at a speed of 500 rpm for 30 seconds. Thereafter, a film was exposed to UV light through a UV irradiator (0.35 mW/cm²) with a total dose of 210 mJ/cm² to prepare a crosslinked film.

### Example 3: Perovskite Compound+Crosslinking Compound 1 (Diazirine-4Bx)

### Preparation of Ink Composition

1.2 mg (20 wt %) of crosslinking compound 1 was added to 1 mL of a FAPbBr₃ perovskite solution (including dodecylamine and an oleic acid ligand) (6 mg/mL in toluene) and stirred.

### Preparation of Blended Film

A prepared ink composition was applied onto glass and then spin-coated at a speed of 500 rpm for 30 seconds.

### Preparation of Crosslinked Film

The prepared ink composition was applied onto glass and then spin-coated at a speed of 500 rpm for 30 seconds. Thereafter, a film was exposed to UV light through a UV irradiator (0.35 mW/cm²) with a total dose of 210 mJ/cm² to prepare a crosslinked film.

### Example 4-1: Perovskite Compound+Crosslinking Compound 1 (Diazirine-4Bx)

### (1) Preparation of Ink Composition

10 wt % of crosslinking compound 1 was added to 1 mL of a FAPbBr₃ perovskite solution (including dodecylamine and an oleic acid ligand) (6 mg/mL in toluene) and stirred.

### Preparation of Blended Film

A prepared ink composition was applied onto glass and then spin-coated at a speed of 500 rpm for 30 seconds.

### Preparation of Crosslinked Film

The prepared ink composition was applied onto glass and then spin-coated at a speed of 500 rpm for 30 seconds. Thereafter, a film was exposed to UV light through a UV irradiator (0.35 mW/cm²) with a total dose of 210 mJ/cm² to prepare a crosslinked film.

### Example 4-2: Perovskite Compound+Crosslinking Compound 2 (Diazirine-6Bx)

### (1) Preparation of Ink Composition

An ink composition was prepared in the same manner as in Example 4-1, except that crosslinking compound 2 was used.

### Preparation of Blended Film

The prepared ink composition was applied onto glass and then spin-coated at a speed of 500 rpm for 30 seconds.

### Preparation of Crosslinked Film

The prepared ink composition was applied onto glass and then spin-coated at a speed of 500 rpm for 30 seconds. Thereafter, a film was exposed to UV light through a UV irradiator (0.35 mW/cm²) with a total dose of 210 mJ/cm² to prepare a crosslinked film.

### Comparative Example 1: Perovskite Compound+Decylamine Ligand

### (1) Preparation of Ink Composition

An ink composition was prepared in the same manner as in Example 1-1, except that a crosslinking compound was not treated.

### (2) Preparation of Blended Film

A prepared ink composition was applied onto glass and then spin-coated at a speed of 500 rpm for 30 seconds.

### Comparative Example 2: Perovskite Compound+Tributylphosphine Oxide Ligand

### (1) Preparation of Ink Composition

An ink composition was prepared in the same manner as in Example 1-2, except that a crosslinking compound was not treated.

### (2) Preparation of Blended Film

The prepared ink composition was applied onto glass and then spin-coated at a speed of 500 rpm for 30 seconds.

### Comparative Example 3: Perovskite Compound+Azide-4Bx

### Preparation of Ink Composition

1.2 mg (20 wt %) of azide-4Bx was added to 1 mL of a FAPbBr₃ perovskite solution (including dodecylamine and an oleic acid ligand) (6 mg/mL in toluene) and stirred.

### Preparation of Blended Film

A prepared ink composition was applied onto glass and then spin-coated at a speed of 500 rpm for 30 seconds.

### Preparation of Crosslinked Film

The prepared ink composition was applied onto glass and then spin-coated at a speed of 500 rpm for 30 seconds. Thereafter, a film was exposed to UV light through a UV irradiator (0.35 mW/cm²) with a total dose of 210 mJ/cm² to prepare a crosslinked film.

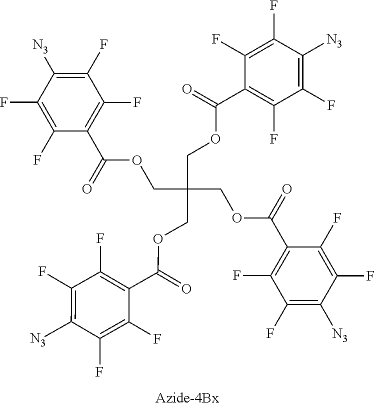 [SMILES: [N-]=[N+]=Nc1c(F)c(F)c(C(=O)OCC(COC(=O)c2c(F)c(F)c(N=[N+]=[N-])c(F)c2F)(COC(=O)c2c(F)c(F)c(N=[N+]=[N-])c(F)c2F)COC(=O)c2c(F)c(F)c(N=[N+]=[N-])c(F)c2F)c(F)c1F]

### Comparative Example 4: Perovskite Compound

### (1) Preparation of Ink Composition

An ink composition was prepared in the same manner as in Example 4-1, except that a crosslinking compound was not used.

### (2) Preparation of Blended Film

The prepared ink composition was applied onto glass and then spin-coated at a speed of 500 rpm for 30 seconds.

### Evaluation Example 1: Confirmation of Properties of Blended Films According to Ligands

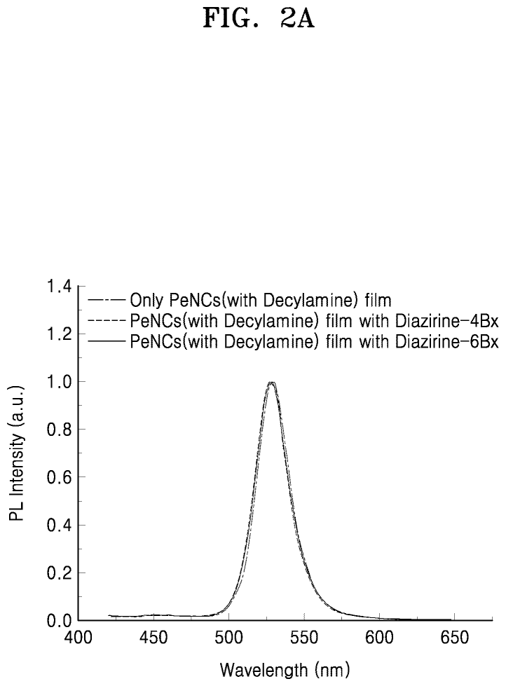

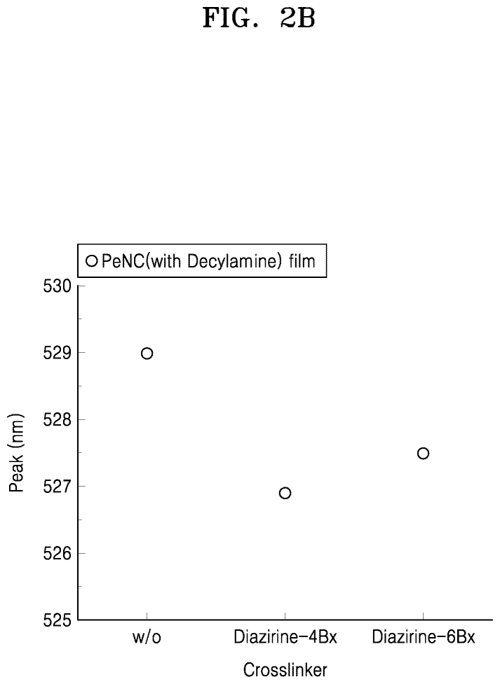

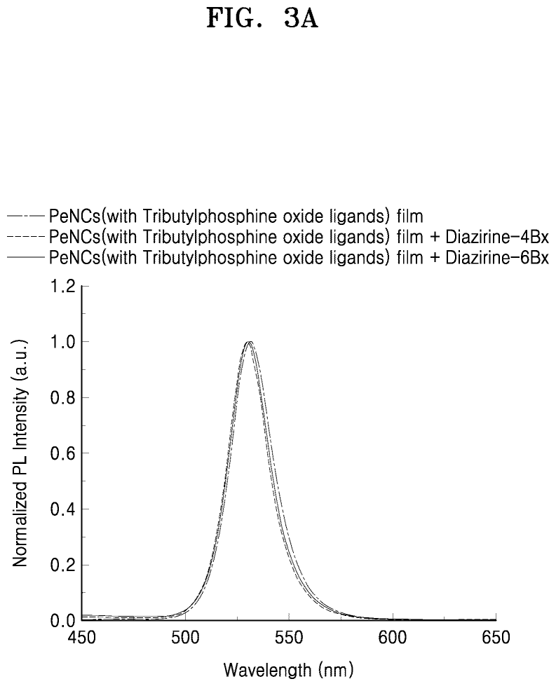

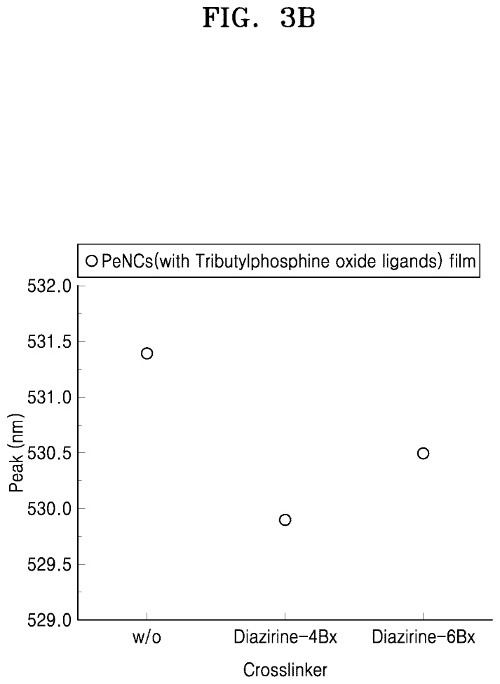

The properties of the blended films according to ligands in Example 1-1, Example 1-2, Example 2-1, Example 2-2, Comparative Example 1, and Comparative Example 2 were confirmed by using a JASCO FP8500 spectrofluorometer. Results thereof are shown in FIGS. 2A to 2B and FIGS. 3A to 3B.

Referring to FIGS. 2A and 2B and 3A and 3B, it was confirmed that, even when different ligands were used, the crosslinking compound according to the disclosure was well mixed with a perovskite compound and thus had excellent compatibility.

In particular, it was confirmed that, even when the crosslinking compound according to the disclosure was mixed, since PL peak shift rarely occurred, the luminescence properties of the perovskite compound did not change.

### Evaluation Example 2: Confirmation of Properties of Blended Films and Crosslinked Films

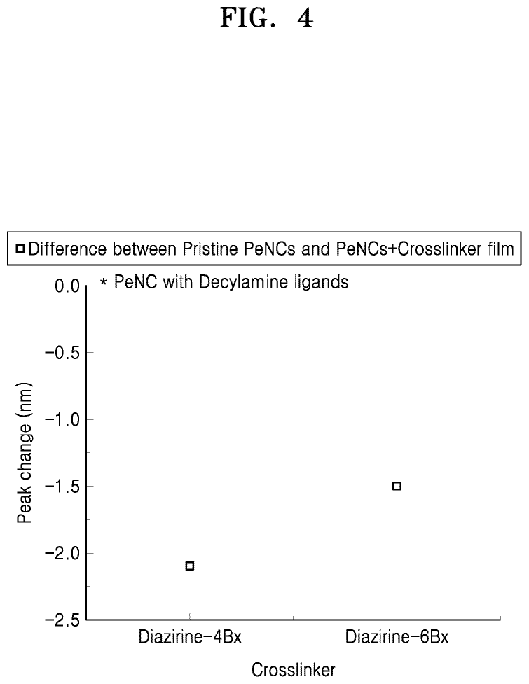

The PL properties of the crosslinked films in Examples 1-1 and 2-1 and the blended film in Comparative Example 1 were confirmed by using a JASCO FP8500 spectrofluorometer, and PL peak shift values of Example 1-1 and Example 2-1 were calculated based on a PL peak of Comparative Example 1 and shown in FIG. 4.

Referring to FIG. 4, it was confirmed that, even when the crosslinking compound according to the disclosure is mixed and crosslinked, since there was no significant change in PL peak, the luminescence properties of perovskite could be maintained.

### Evaluation Example 3: Confirmation of PL Intensity Properties According to Crosslinking Time

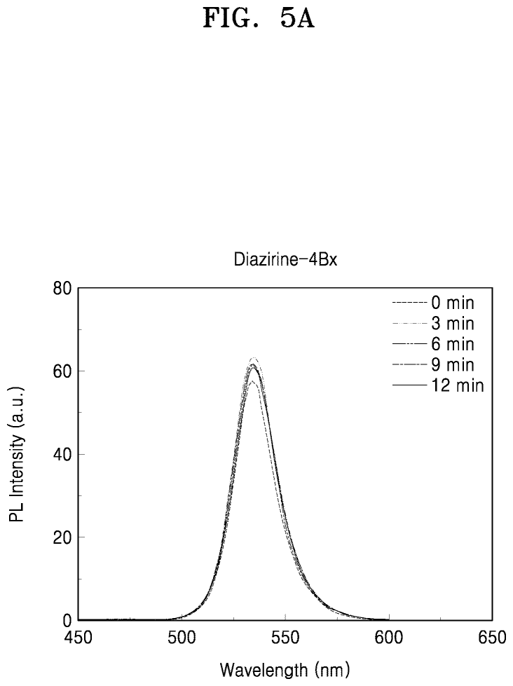

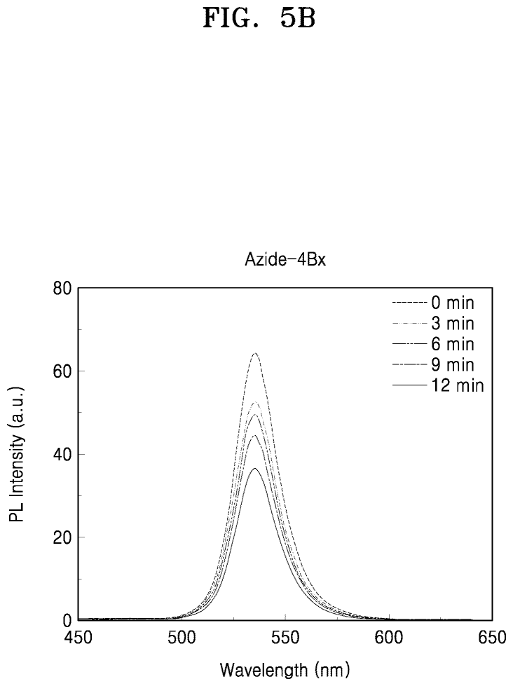

The PL intensity properties of the crosslinked films according to crosslinking time in Example 3 and Comparative Example 3 were confirmed by using a JASCO FP-8500 spectrofluorometer. Results thereof are shown in FIGS. 5A and 5B.

Referring to FIGS. 5A and 5B, it was confirmed that a PL intensity value of the crosslinked film of Example 3 was maintained constant according to crosslinking time (that is, a UV exposure time), but a PL intensity value of the crosslinked film of Comparative Example 3 was significantly decreased.

Therefore, it was confirmed that the crosslinked film including the crosslinking compound according to the disclosure was minimized from being damaged during a patterning process, and luminescence properties were maintained.

### Evaluation Example 4: Confirmation of PL Intensity Properties of Ink Composition

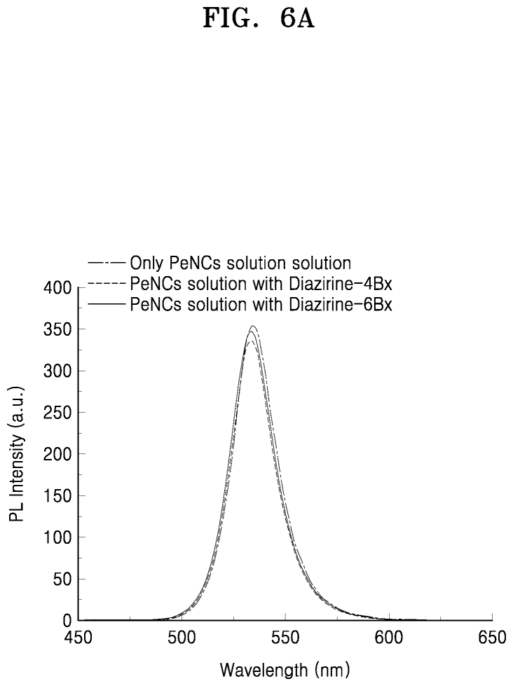

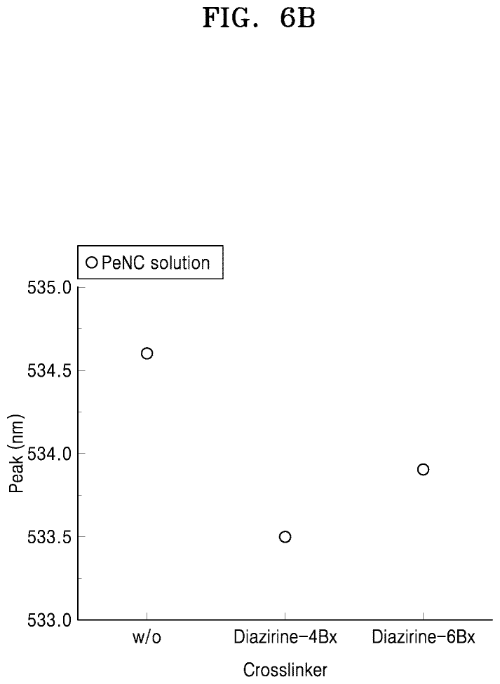

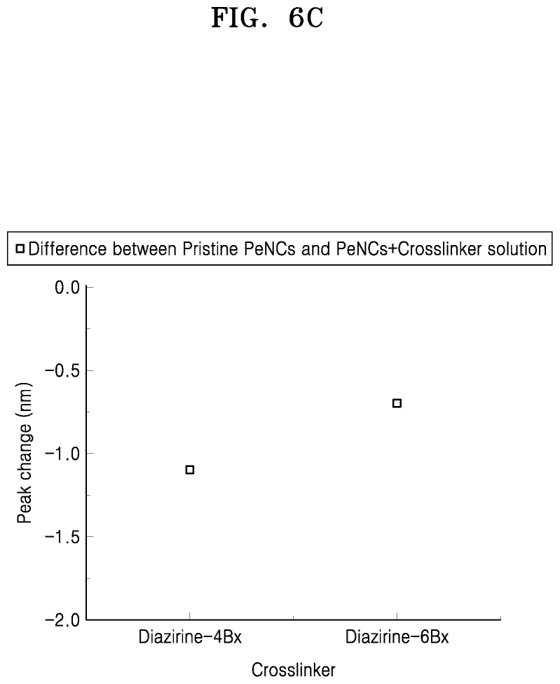

The PL intensity properties of the ink compositions of Example 4-1, Example 4-2, and Comparative Example 4 were confirmed by using a JASCO FP8500 spectrofluorometer. Results thereof are shown in FIGS. 6A and 6B. FIG. 6C shows PL peak shift values of Example 4-1 and Examples 4-2 with respect to a PL peak of Comparative Example 4.

Referring to FIGS. 6A to 6C, it was confirmed that, even when the crosslinking compound according to the disclosure was mixed, since there was no significant change in PL peak, the crosslinking compound had excellent compatibility with a perovskite compound, and the luminescence properties of perovskite could be maintained.

### Evaluation Example 5: Confirmation of Properties of Film

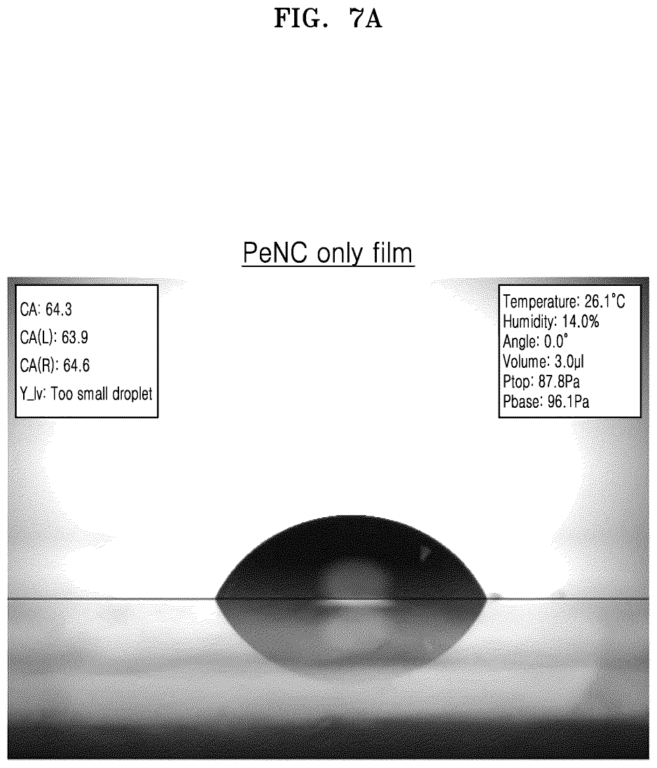

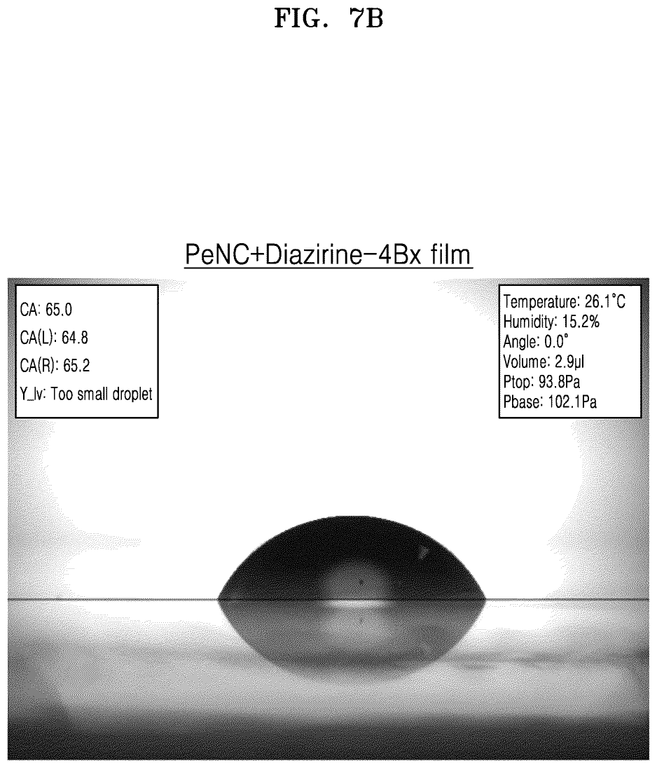

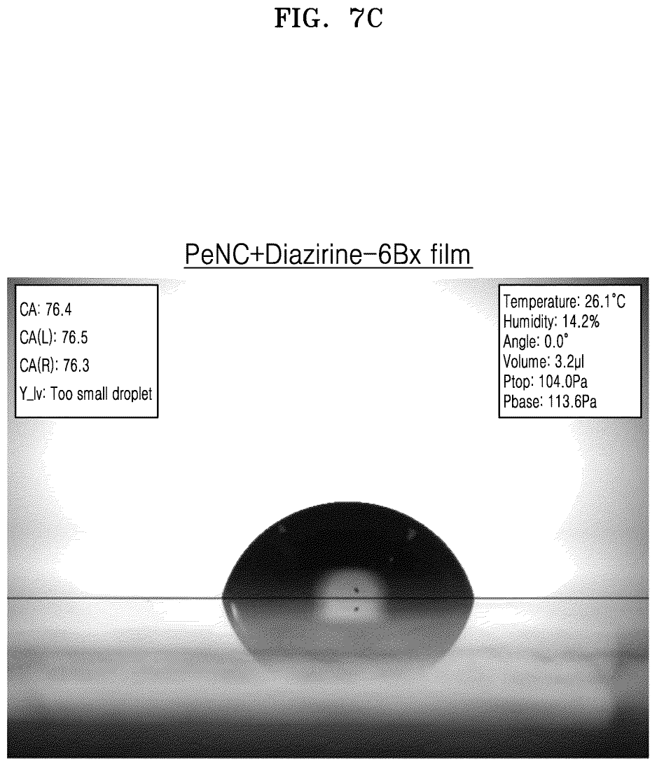

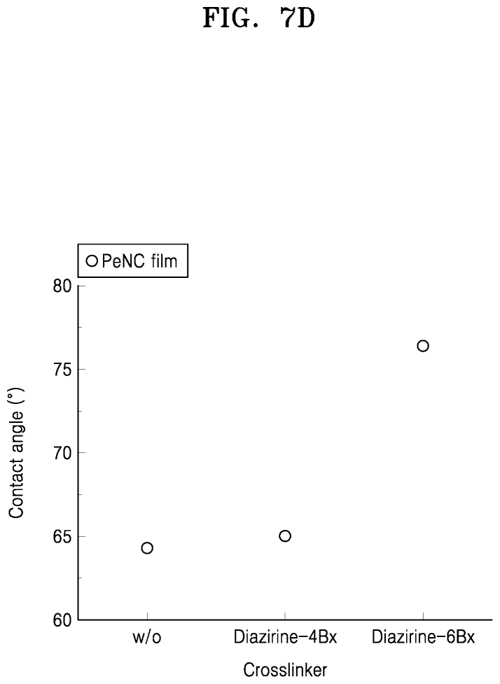

The properties of the crosslinked films of Example 4-1, Example 4-2, and Comparative Example 4 were confirmed by using Smart drop equipment (manufactured by Femtobiomed Inc.). Results thereof are shown in FIGS. 7A to 7D. A contact angle was measured by dropping a water droplet onto a blended film.

Referring to FIGS. 7A to 7D, it was confirmed that, as a contact angle of a crosslinked film prepared by using a crosslinking compound was significantly increased, the hydrophobicity of a film surface was improved, resulting in improvement in moisture stability. In addition, it was confirmed that, as a size of the crosslinking compound increased, the hydrophobicity was increased, thereby improving moisture stability.

### Evaluation Example 6: Confirmation of Crosslinking Efficiency According to UV Absorption Properties According to Crosslinking Time

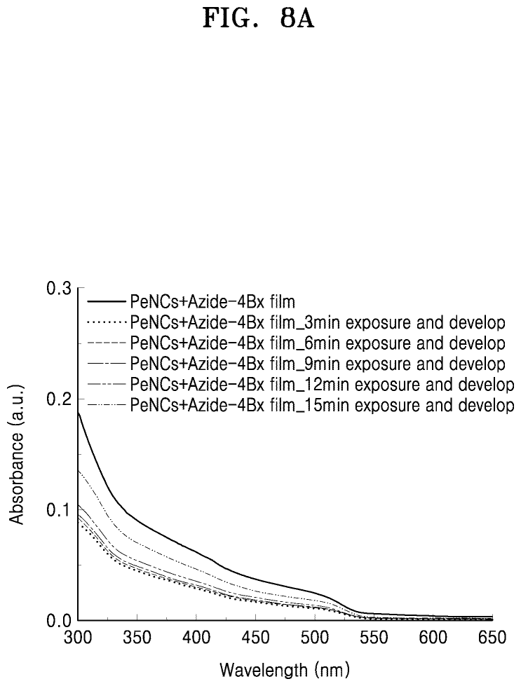

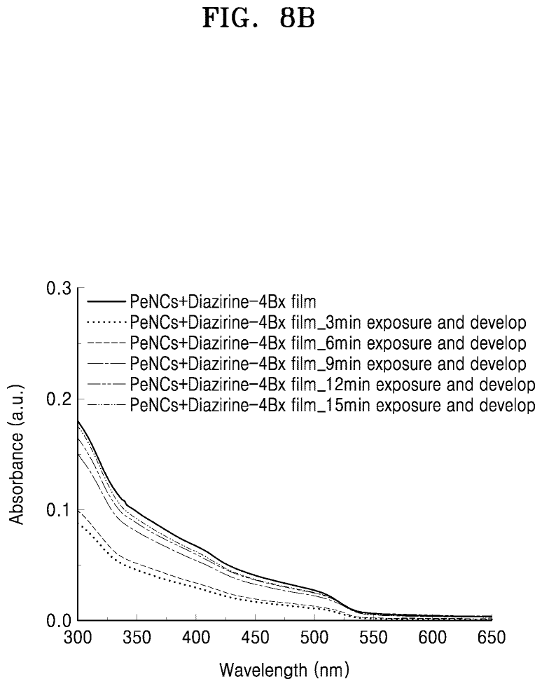

Crosslinking efficiency according to UV absorption properties of Example 3 and Comparative Example 3 according to crosslinking time was confirmed. Results thereof are shown in FIGS. 8A and 8B. In this case, the ink compositions of Example 3 and Comparative Example 3 were spin-coated to prepare blended films, and then an exposure time was increased at an interval of 3 minutes through a UV irradiator (0.35 mW/cm²) and adjusted. Thereafter, only exposed films were developed by using a non-polar solvent, and the corresponding films were measured by using a Lambda-465 UV-vis spectrophotometer.

Referring to FIGS. 8A and 8B, a crosslinked film including the crosslinking compound of Comparative Example 3 was not completely crosslinked even after 15 minutes, but a film including the crosslinking compound according to Example 3 of the disclosure was confirmed to be crosslinked within a short period of time, thereby confirming that the crosslinking efficiency of the crosslinking compound of the disclosure was superior to that of the Comparative Example.

Since a crosslinking compound according to the disclosure has excellent compatibility and miscibility with materials for electronic devices, a film including the crosslinking compound according to the disclosure may maintain the optical and electrical properties of a material.

While the disclosure has been described with the above embodiments, this is merely illustrative, and those skilled in the art will understand that various modifications and other equivalent embodiments are possible therefrom. Therefore, the true technical scope of the disclosure should be defined by the technical spirit of the appended claims

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope of the disclosure as defined by the following claims.

1. A crosslinking compound represented by Formula 1 below: 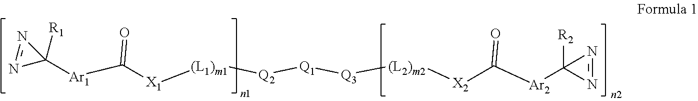
wherein, in Formula 1,
L₁ and L₂ are each independently a single bond or a C₁-C₃₀ alkylene group unsubstituted or substituted with at least one R₂₀,
m1 and m2 are each independently 0, 1, 2, 3, 4, 5, or 6,
Ar₁ and Ar₂ are each a C₅-C₆₀ carbocyclic group unsubstituted or substituted with at least one R₂₀ or a C₁-C₆₀ heterocyclic group unsubstituted or substituted with at least one R₂₀,
n1 and n2 are each independently 0, 1, 2, 3, 4, 5, 6, 7, 8, 9, or 10,
the sum of n1 and n2 is greater than or equal to 2,
Q₁ to Q₃ are each a single bond, O, S, C, C(R₃), C(R₃)(R₄), N, N(R₅), P, P(R₅)(R₆)(R₇), P=O(R₈), SO₂, a C₁-C₃₀ alkylene group unsubstituted or substituted with at least one R₂₀, or any combination thereof,
X₁ is O, S, Se, N(R₉), or C(R₉)(R₁₀),
X₂ is O, S, Se, N(R₁₁), or C(R₁₁)(R₁₂),
R₁ to R₁₂ and R₂₀ are each independently hydrogen, deuterium, —CF₃, —CCl₃, —F, —Cl, —Br, —I, a hydroxyl group, a cyano group, a C₁-C₃₀ alkyl group, a C₂-C₃₀ alkenyl group, a C₂-C₃₀ alkynyl group, a C₁-C₃₀ alkoxy group, a C₁-C₃₀ alkylthio group, a C₅-C₆₀ carbocyclic group, a C₁-C₆₀ heterocyclic group, or —Si(Q₁₁)(Q₁₂)(Q₁₃), and
Q₁₁ to Q₁₃ are each independently hydrogen, deuterium, —F, —Cl, —Br, —I, a hydroxyl group, a cyano group, a C₁-C₃₀ alkyl group, a C₂-C₃₀ alkenyl group, a C₂-C₃₀ alkynyl group, a C₁-C₃₀ alkoxy group, or a C₁-C₃₀ alkylthio group.

2. The crosslinking compound of claim 1, wherein L₁ and L₂ are each independently a methylene group, an ethylene group, a propylene group, or a butylene group, each unsubstituted or substituted with at least one R₂₀.

3. The crosslinking compound of claim 1, wherein L₁ and L₂ are each independently a methylene group unsubstituted or substituted with at least one R₂₀.

4. The crosslinking compound of claim 1, wherein m1 is 1, and
m2 is 1.

5. The crosslinking compound of claim 1, wherein Ar₁ and Ar₂ are each independently a benzene group, a naphthalene group, an anthracene group, a phenanthrene group, a triphenylene group, a pyrene group, a chrysene group, a cyclopentadiene group, a furan group, a thiophene group, a silole group, an indene group, a fluorene group, an indole group, a carbazole group, a benzofuran group, a dibenzofuran group, a benzothiophene group, a dibenzothiophene group, a benzosilole group, a dibenzosilole group, an aza-fluorene group, an aza-carbazole group, an aza-dibenzofuran group, an aza-dibenzothiophene group, an aza-dibenzosilole group, a pyridine group, a pyrimidine group, a pyrazine group, a pyridazine group, a triazine group, a quinoline group, an isoquinoline group, a quinoxaline group, a quinazoline group, a phenanthroline group, a pyrrole group, a pyrazole group, an imidazole group, a triazole group, an oxazole group, an isoxazole group, a thiazole group, an isothiazole group, an oxadiazole group, a thiadiazole group, a benzopyrazole group, a benzoimidazole group, a benzoxazole group, a benzothiazole group, a benzoxadiazole group, a benzothiadiazole group, a dibenzooxasiline group, a dibenzothiasiline group, a dibenzodihydroazasiline group, a dibenzodihydrodisiline group, a dibenzodihydrosiline group, a dibenzodioxin group, a dibenzooxathine group, a dibenzooxazine group, a dibenzopyran group, a dibenzodithine group, a dibenzothiazine group, a dibenzothiopyran group, a dibenzocyclohexadiene group, a dibenzodihydropyridine group, or a dibenzodihydropyrazine group, each unsubstituted or substituted with at least one R₂₀.

6. The crosslinking compound of claim 1, wherein Ar₁ and Ar₂ are each independently a benzene group, a naphthalene group, a phenanthrene group, a pyrene group, or an anthracene group, each unsubstituted or substituted with at least one R₂₀.

7. The crosslinking compound of claim 1, wherein X₁ is O, N(R₉), S, or Se.

8. The crosslinking compound of claim 1, wherein X₂ is O, N(R₁₁), S, or Se.

9. The crosslinking compound of claim 1, wherein X₁ and X₂ are each independently O or S.

10. The crosslinking compound of claim 1, wherein Q₁ is C or O.

11. The crosslinking compound of claim 1, wherein R₁ to R₁₂ and R₂₀ are each independently: hydrogen, deuterium, —CF₃, —CCl₃, —F, —Cl, —Br, —I, a hydroxyl group, a cyano group, a C₁-C₂₀ alkyl group, or a C₁-C₂₀ alkoxy group; a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a biphenyl group, a terphenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, benzoimidazolyl group, a benzofuranyl group, a benzo thiophenyl group, a benzoisothiazolyl group, a benzoxazolyl group, a benzoisoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, an imidazopyrimidinyl group, an azacarbazolyl group, an azadibenzofuranyl group, an azadibenzothiophenyl group, an azafluorenyl group, or an azadibenzosilolyl group; or —Si(Q₁₁)(Q₁₂)(Q₁₃), and
Q₁₁ to Q₁₃ are each independently hydrogen, deuterium, —F, —Cl, —Br, —I, a hydroxyl group, a cyano group, a C₁-C₃₀ alkyl group, a C₂-C₃₀ alkenyl group, a C₂-C₃₀ alkynyl group, a C₁-C₃₀ alkoxy group, or a C₁-C₃₀ alkylthio group.

12. The crosslinking compound of claim 1, wherein R₁ to R₁₂ and R₂₀ are each independently: hydrogen, deuterium, —CF₃, —CCl₃, or a C₁-C₂₀ alkyl group; a cyclopentenyl group, a cyclohexenyl group, a cycloheptenyl group, a phenyl group, a biphenyl group, a terphenyl group, a naphthyl group, a fluorenyl group, a phenanthrenyl group, anthracenyl group, a fluoranthenyl group, a triphenylenyl group, a pyrenyl group, a chrysenyl group, or a pyridinyl group; or —Si(Q₁₁)(Q₁₂)(Q₁₃), and
Q₁₁ to Q₁₃ are each independently hydrogen, deuterium, —F, —Cl, —Br, —I, a hydroxyl group, a cyano group, a C₁-C₃₀ alkyl group, a C₂-C₃₀ alkenyl group, a C₂-C₃₀ alkynyl group, a C₁-C₃₀ alkoxy group, or a C₁-C₃₀ alkylthio group.

13. The crosslinking compound of claim 1, wherein R₁ and R₂ are each independently hydrogen, deuterium, —CF₃, —CCl₃, —F, —Cl, —Br, —I, a hydroxyl group, a cyano group, or a C₁-C₃₀ alkyl group.

14. The crosslinking compound of claim 1, wherein R₁ and R₂ are each independently —CF₃.

15. The crosslinking compound of claim 1, wherein the crosslinking compound is represented by Formula 2 below: 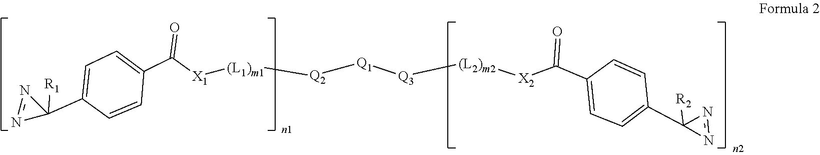
wherein, in Formula 2, L₁, L₂, m1, m2, n1, n2, Q₁ to Q₃, X₁, X₂, R₁, and R₂ are as defined claim 1.

16. The crosslinking compound of claim 1, wherein the crosslinking compound is represented by Formula 3-1 or 3-2 below: 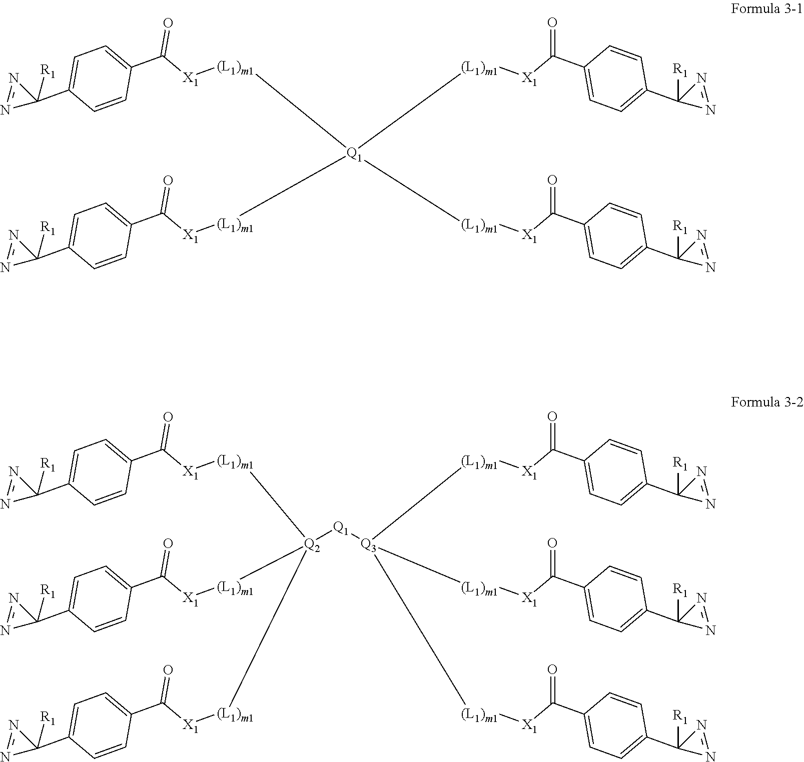
wherein, in Formulas 3-1 and 3-2, L₁, L₂, m1, m2, X₁, X₂, R₁, and R₂ are as defined in claim 1,
in Formula 3-1, Q₁ is C or a C₁-C₃₀ alkylene group unsubstituted or substituted with at least one R₂₀,
in Formula 3-2, Q₁ is as defined in claim 1, and
in Formula 3-2, Q₂ and Q₃ are each independently C, a C₁-C₃₀ alkylene group unsubstituted or substituted with at least one R₂₀, or a combination of C(R₃)(R₄) and C.

17. The crosslinking compound of claim 1, wherein the crosslinking compound is represented by Formula 4-1 or 4-2 below: 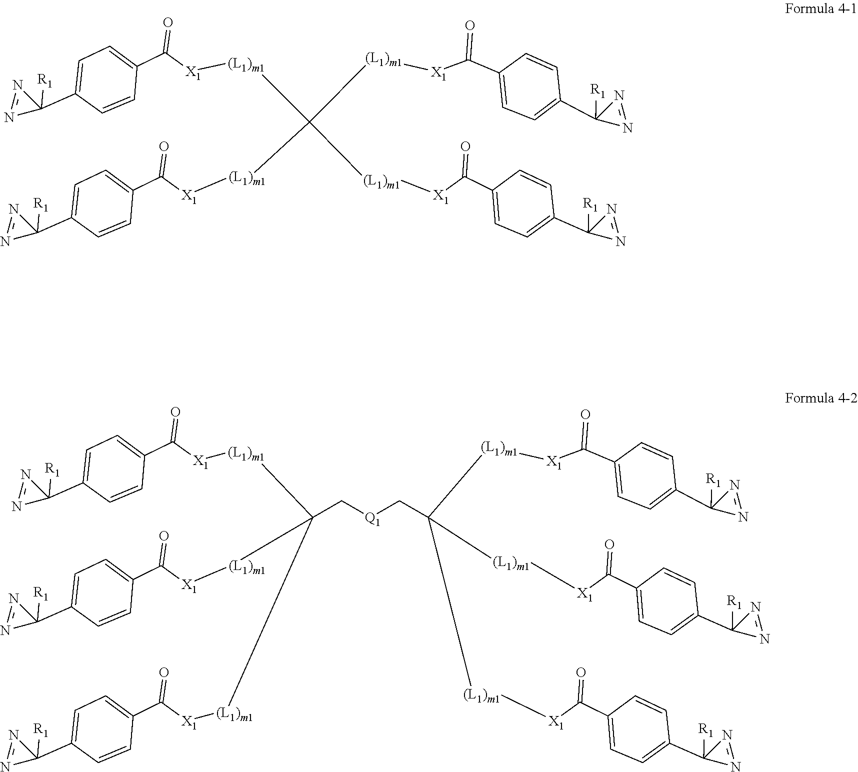
wherein, in Formulas 4-1 and 4-2, L₁, m1, X₁, and Q₁ are as defined in claim 1.

18. The crosslinking compound of claim 1, wherein the crosslinking compound is compound 1 or 2 below:

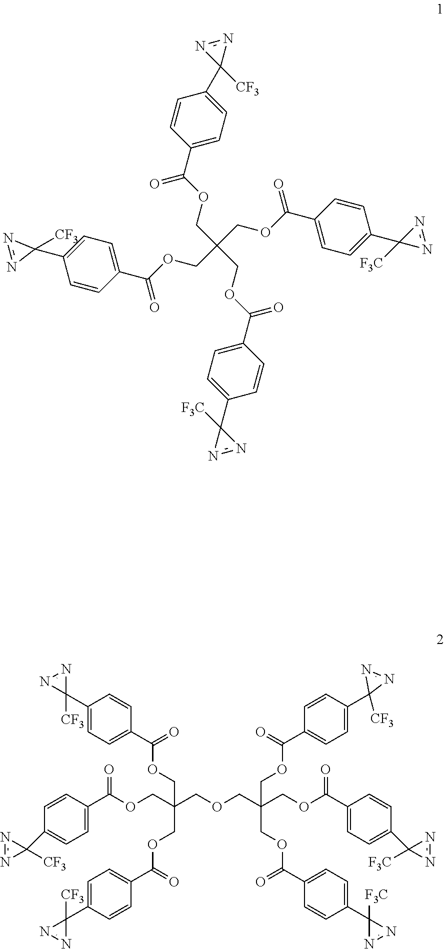 [SMILES: O=C(OCC(COC(=O)c1ccc(C2(C(F)(F)F)N=N2)cc1)(COC(=O)c1ccc(C2(C(F)(F)F)N=N2)cc1)COC(=O)c1ccc(C2(C(F)(F)F)N=N2)cc1)c1ccc(C2(C(F)(F)F)N=N2)cc1 ; O=C(OCC(COCC(COC(=O)c1ccc(C2(C(F)(F)F)N=N2)cc1)(COC(=O)c1ccc(C2(C(F)(F)F)N=N2)cc1)COC(=O)c1ccc(C2(C(F)(F)F)N=N2)cc1)(COC(=O)c1ccc(C2(C(F)(F)F)N=N2)cc1)COC(=O)c1ccc(C2(C(F)(F)F)N=N2)cc1)c1ccc(C2(C(F)(F)F)N=N2)cc1]

19. A film comprising a crosslinking compound, wherein the crosslinking compound is represented by Formula 1 below: 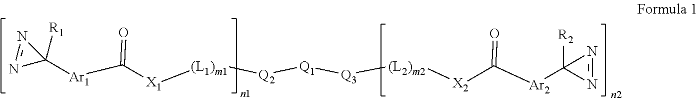
wherein, in Formula 1,
L₁ and L₂ are each independently a single bond or a C₁-C₃₀ alkylene group unsubstituted or substituted with at least one R₂₀,
m1 and m2 are each independently 0, 1, 2, 3, 4, 5, or 6,
Ar₁ and Ar₂ are each a C₅-C₆₀ carbocyclic group unsubstituted or substituted with at least one R₂₀ or a C₁-C₆₀ heterocyclic group unsubstituted or substituted with at least one R₂₀,
n1 and n2 are each independently 0, 1, 2, 3, 4, 5, 6, 7, 8, 9, or 10,
the sum of n1 and n2 is greater than or equal to 2, Q₁ to Q₃ are each a single bond, O, S, C, C(R₃), C(R₃)(R₄), N, N(R₅), P, P(R₅)(R₆)(R₇), P=O(R₈), SO₂, a C₁-C₃₀ alkylene group unsubstituted or substituted with at least one R₂₀, or any combination thereof,
X₁ is O, S, Se, N(R₉), or C(R₉)(R₁₀),
X₂ is O, S, Se, N(R₁₁), or C(R₁₁)(R₁₂),
R₁ to R₁₂ and R₂₀ are each independently hydrogen, deuterium, —CF₃, —CCl₃, —F, —Cl, —Br, —I, a hydroxyl group, a cyano group, a C₁-C₃₀ alkyl group, a C₂-C₃₀ alkenyl group, a C₂-C₃₀ alkynyl group, a C₁-C₃₀ alkoxy group, a C₁-C₃₀ alkylthio group, a C₅-C₆₀ carbocyclic group, a C₁-C₆₀ heterocyclic group, or —Si(Q₁₁)(Q₁₂)(Q₁₃), and
Q₁₁ to Q₁₃ are each independently hydrogen, deuterium, —F, —Cl, —Br, —I, a hydroxyl group, a cyano group, a C₁-C₃₀ alkyl group, a C₂-C₃₀ alkenyl group, a C₂-C₃₀ alkynyl group, a C₁-C₃₀ alkoxy group, or a C₁-C₃₀ alkylthio group.

20. The film of claim 19, further comprising a substrate.
